(19) United States
(12) Patent Application Publication
(10) Pub. No.: US 2014/0222374 A1
(43) Pub. Date: Aug. 7, 2014

(54) System and Method for Measuring Parameters Relating to Agriculture

(71) Applicant: MONFORD AG SYSTEMS LIMITED, co. Wicklow Newcastle (IE)

(72) Inventors: Steven Lock, New Castle (IE); David Guerin, New Castle (IE); Jake Byrne, New Castle (IE); Jerrold C. Manock, New Castle (IE)

(21) Appl. No.: 14/345,805

(22) PCT Filed: Sep. 20, 2012
(86) PCT No.: PCT/EP2012/068565

§ 371 Date: Mar. 19, 2014

(30) Foreign Application Priority Data
Sep. 20, 2011 (GB) 1116194.0

(51) Int. Cl. G01N 33/00

(52) U.S. Cl. CPC G01N 33/0098
USPC 702/166

(57) ABSTRACT

The invention provides a system and method system for measuring agricultural parameters, for example the quantity of an agricultural product such as grass. The system comprising means for triggering a measurement of data and repeating the measurement at a plurality of locations over an area; means for communicating the measurement data to a processing means; and means for calculating the quantity of agricultural product for the area using the processing means. The invention solves a problem of how to effectively and simply measure an agricultural product, such as grass, and provides a benefit by allowing the farmer to quickly and accurately measure their grass in a particular field or area.

### FIELD OF THE INVENTION

The invention relates to a system and method for measuring parameters relating to agriculture, for example the quantity of an agricultural product. In particular, the invention relates to a system and method for measuring the quantity of grass in a predefined area.

### BACKGROUND TO THE INVENTION

Grass measurement started in the New Zealand dairy industry and is now acknowledged as industry best practice, and promoted in all countries with a grass-based farming system. There is a renewed interest in grazing systems in many temperate and subtropical regions of the world (especially Europe, South America, New Zealand, Australia and USA), as a consequence of lower inflation-adjusted prices, the proposed removal of some subsidies and tariffs, and rising labor, machinery and housing costs. In addition trends have favored agricultural products that are produced sustainably or naturally.

Research shows that use of grass for grazing should provide the basis of sustainable livestock systems, as grazed grass is the cheapest source of nutrients for ruminants. Indeed, the competitiveness of grass as a feed is unparalleled. In that context, a key objective for grazing systems is to ensure high grass use, thus allowing increased output per hectare for all sectors.

Research from Teagasc, a state agricultural body based in Ireland, has determined that the financial benefit of measuring grass amounts to approximately [symbol] 120 per cow per annum, which equates to [symbol] 12,000 per year for a herd of 100 cows. In 2009, according to a National Farm Survey, top farms measuring grass used 30% more grass than the average farm, allowing them a stocking rate of 2.69 cows per hectare compared to the national average of 1.87. These farms used 25% less concentrates, and milk solids in kilograms per hectare were 36% higher. Where grass is measured in kilograms of dry matter per hectare (kg DM/ha), it is estimated that one cow consumes 17 kg DM per day to produce 25 liters of milk.

Research shows that leaf production is maximized by grazing to between 3.5 and 4 cm of residual height. With good quality grass, this is expected to yield 1250 kg DM/ha. By keeping the pasture in a growing state, a higher quality of grass will be produced in a green leafy base. Pre-grazing height should be between 8 and 9 cm, corresponding to three leaves approximately. If such grass is grazed down to between 3.5 and 4 cm, growth will be expected at 16,000 kg DM/ha.

While every cattle farmer should aim to provide his herd with excellent quality grass throughout the grazing season, this is not always easy to achieve. A core problem faced by many farmers, is to determine whether the quantity of grass they have available for grazing, meets or will meet requirements of their herds.

There are a number of techniques available for measuring grass. A first example comprises simply Cutting, Drying, Weighing Shears, Quadrant, Scales. However this method is slow and laborious. Another substantially mechanical system is a Rising Plate Meter, for instance as disclosed in New Zealand patent 286786. However, examples according to this technique are cumbersome to carry and to use accurately.

More recently, electronic system have been developed, which rely upon ultrasonic devices for carrying out measurements. Examples of such systems re disclosed in patent applications WO2006009472, WO2008151371 and US2003004630. However, all such systems require an extensive amount of components with correspondingly high power requirements and complicated architectures, as well as extensive user input requirements for their use.

It is an object of the invention to provide a system and a method for measuring the quantity of an agricultural product or an agricultural parameter, which overcomes at least one of the above problems.

### SUMMARY OF THE INVENTION

According to a first aspect of the invention, there is provided, as set out in the appended claims, a system for measuring a quantity of an agricultural product, comprising means for measuring agricultural product data; an accelerometer for triggering the measuring means and repeating the measurement at a plurality of locations over an area; means for communicating the measurement data to a remote processing means; and means for calculating the quantity of agricultural product for the area using the processing means.

The invention solves the problem of how to effectively and simply measure an agricultural product, such as grass, kale or other crop, and usefully allows a user, typically a farmer, to quickly and accurately measure the grass in a particular field or area. The system of the invention advantageously combines a number of advantages, which include a substantially hands-free and input-free actuation, a high sampling rate, accuracy of measurement, ability to share data, all resulting in simplicity of use, added convenience and time-saving for the user.

In an embodiment of the system according to the invention, the accelerometer and the communicating means are contained in a primary module having a rechargeable power source. In a variant of this embodiment, the means for measuring data are preferably contained in a secondary module drawing power from the primary module. Preferably, the primary module is configured to releasably retain the secondary module in use. Alternatively, the primary module may be configured to releasably retain a plurality of secondary modules in use.

In an embodiment of the system according to the invention, the means for measuring data is preferably selected from the group comprising remote and contact sensors including but not limited to an ultrasonic ranging device, a luminosity measuring device, a photosynthetically active radiation meter, a spectrometer, a moisture meter, a temperature meter, a ph meter, a nitrogen sensor, a phosphate sensor, a sulphur sensor, a lime sensor, a methane and other gas sensors and other sensors to be determined. In a variant light detection ranging sensors can be used for example optical and laser sensors using ultraviolet, visible and near infrared light, photodetectors and electromagnetic sensors. In another variant of this embodiment, at least one ultrasonic ranging device may be adapted to measure a plant height in the range 0 to 1.5 cm, or in the range 1.5 to 30 cm, or in the range 30 to 150 cm.

In an embodiment of the system according to the invention, the means for communicating the measurement data is selected from the group comprising a near field communication device according to the ISO 13157 networking standard, a wireless data transmitter conforming to the IEEE 802.15.1 Bluetooth networking standard, and a wireless data transmitter conforming to the IEEE 802.11 WiFi networking standard.

In an embodiment of the system according to the invention, the remote processing means is selected from the group comprising computers, portable computers, tablet computers, mobile telephone handsets.

In an embodiment of the system according to the invention, the remote processing means preferably comprises means for associating measurement data with coordinate data. In a variant of this embodiment, the remote processing means may further comprise means for mapping coordinate data and measurement data to define a measured area. The remote processing means may advantageously further comprise means for mapping associated measurement data and coordinate data on a map and, in a particularly useful variant of this embodiment, the system may further comprise an interface for visually representing the measured data on a colour-coded or gray-scale map. In any of these embodiments, the coordinate may comprise at least one Global Positioning System coordinate.

In an embodiment of the system according to the invention, the system may further comprise a mounting bracket for mounting the system to a support, at a height substantially above the agricultural product to be measured. The mounting bracket is preferably one suitable for a support selected from the group comprising user footwear, a walking aid, a user vehicle, a post, a gate, a fence, a mobile robot, a drone aerial vehicle, an animal.

The agricultural product preferably comprises grass, as may for instance be found in a grazing field, a golf course, sports stadium or a park area. It will be appreciated that other produces can be measured such as kale and other crops.

According to another aspect of the invention, there is provided a method for measuring a quantity of an agricultural product, comprising the steps of measuring the agricultural product upon receipt of a measurement trigger provided by an accelerometer; repeating the measurement trigger at a plurality of locations over an area; communicating the measurement data to a remote processing means; and calculating the quantity of agricultural product for the area at the remote processing means.

In an embodiment of the system according to the invention, the step of measuring is preferably performed by at least one selected from the group comprising an ultrasonic ranging device, a luminosity measuring device, a photosynthetically active radiation meter and a spectrometer. In a variant of this embodiment, the step of measuring performed by the at least one ultrasonic ranging device may comprise the further step of measuring a plant height in the range 0 to 1.5 cm, or in the range 1.5 to 30 cm, or in the range 30 to 150 cm.

In an embodiment of the system according to the invention, the step of communicating the measurement data may be performed by one selected from the group comprising a near field communication device according to the ISO 13157 networking standard, a wireless data transmitter conforming to the IEEE 802.15.1 Bluetooth networking standard, a wireless data transmitter conforming to the IEEE 802.11 WiFi networking standard.

In an embodiment of the system according to the invention, the method may comprise the further step of associating measurement data with coordinate data. In a variant of this embodiment, the method may comprise the further step of mapping coordinate data and measurement data to define an measured area. In either of these embodiments, the remote processing means may further comprise means for mapping associated measurement data and coordinate data on a map. In a particularly useful variant of this embodiment, the method may comprise the further step of visually representing the quantity of measured agricultural product on a colour-coded map in an interface. For any of these embodiments, the coordinate preferably comprises at least one Global Positioning Method coordinate.

In an embodiment of the system according to the invention, the remote processing means is selected from the group comprising computers, portable computers, tablet computers, mobile telephone handsets.

In an embodiment of the system according to the invention, the method may comprise the further step of mounting the system to a support, at a height substantially above the agricultural product to be measured.

In a further embodiment of the invention there is provided a system for measuring an agricultural parameter, the system comprising:
a primary module comprising a data processing unit, a power supply and coupled with a memory means;
a secondary module comprising a sensor,
means for triggering the sensor to measure an agricultural parameter and repeating the measurement at a plurality of locations over an area;
   means for communicating the measured agricultural parameters to a remote processing means.

According to another aspect of the invention there is provided a system for measuring a quantity of an agricultural product or an agricultural parameter, the system comprising:
means for measuring agricultural product data or agricultural parameters;
means for triggering the measuring means and repeating the measurement at a plurality of locations over an area;
means for communicating the measurement data to a remote processing means; and
means for calculating the quantity of agricultural product for the area or an output of the agricultural parameter using the processing means.

According to yet another aspect of the present invention, there is also provided a set of instructions recorded on a data carrying medium, carrier signal or read-only memory and which, when processed by a data processing terminal having networking means, configures the terminal as the remote processing means substantially as described above. The set of instructions may alternatively configure the terminal to perform the steps of the method substantially as described above.

The set of instructions may be advantageously embodied as an application package file (‘APK’) for use with the Android™ operating system or embodied as an iPhone™ application archive (‘IPA’) for use with the iOS™ operating system.

According to still another aspect of the present invention, there is also provided a kit of parts for measuring a quantity of an agricultural product, comprising a primary module, at least one secondary module and a set of instructions substantially as described above. The kit of parts may usefully further include a mounting bracket substantially as described above.

### BRIEF DESCRIPTION OF THE DRAWINGS

The invention will be more clearly understood from the following description of an embodiment thereof, given by way of example only, with reference to the accompanying drawings, in which:

FIG. 1 illustrates an embodiment of a system according to the invention in use, including an accelerometer-triggered unit and a remote data processing terminal;

FIG. 2 illustrates an embodiment of the accelerometer-triggered unit of FIG. 1, comprising a primary unit and a secondary unit;

FIG. 3 provides a functional representation of the respective components of the primary and secondary units of FIG. 2.

FIG. 4 provides a functional representation of the typical components of the remote data processing terminal of FIG. 1.

FIG. 5 illustrates a further embodiment of a system according to the invention, that may be practiced with the accelerometer-triggered unit and remote data processing terminal system of FIGS. 1 to 4, including geographical and map data for associating with agricultural product measurement data;

FIG. 6 shows a network environment in which the remote data processing terminal of FIGS. 1 to 5 may obtain geographical and map data for associating with agricultural product measurement data and/or share measurement data with further remote data processing terminals; and

FIG. 7 details data processing steps of an embodiment of the method according to the invention, performed by each embodiments of FIGS. 1 to 6 at runtime.

### DETAILED DESCRIPTION OF THE DRAWINGS

There will now be described by way of example several specific modes contemplated by the inventors. In the following description numerous specific details are set forth in order to provide a thorough understanding. It will be apparent however, to one skilled in the art, that the present invention may be practiced without limitation to these specific details. In other instances, well known methods and structures have not been described in detail so as not to unnecessarily obscure the description.

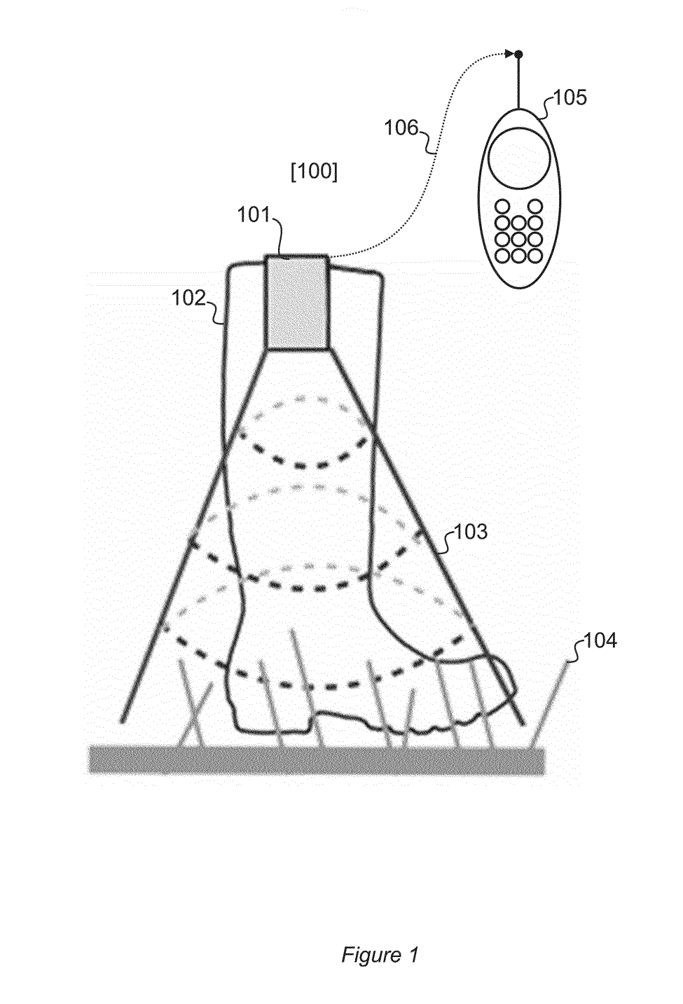

Referring now to the figures and initially FIG. 1, a system 100 for measuring the quantity of an agricultural product 104 is shown, for example grass, according to one embodiment of the invention. The system 100 comprises means for triggering a measurement of data and repeating the measurement at a plurality of locations over an area and means for communicating 106 the measurement data to a processing means 105, embodied in an accelerometer-triggered unit 101; and means for calculating the quantity of agricultural product for the area using the processing means 105.

As shown in FIG. 1 the invention can be attached to a farmer's boot 102, or to a stick. Basically the unit 101 has to be positioned at a height above the agricultural product 104 to be measured. After calibrating on a flat surface, agricultural product measurements 103 are generated utilizing an accelerometer as the farmer (or user) walks around their field or defined area with an ultrasound device. The ultrasound device 101 can be pressure activated or triggered with each step taken by the farmer. The measurement data is then processed by processing means 105, which calculates the quantity of agricultural product for the area. The device 101 using ultrasound can send data to a system software application via Bluetooth 106, or other wireless means or communication means.

As shown in FIG. 1, the system 100 of the invention can provide a combination of a hardware device 101 and a software application on a Smart Phone 105, or other mobile computing device, to allow a farmer to measure the grass 104 in a pasture or area to be measured automatically. In the embodiment shown, triggering a measurement of data and repeating the measurement at a plurality of locations over an area is performed using a ultrasound range finder and accelerometer, and the measurement data is transmitted to a remote communications device 105, for example using a wireless Bluetooth communication 106.

The remote communications device 105 can be a Smart Phone, for example the iPhone™ made by Apple, Inc., or other appropriate mobile computing device, and comprises the processing means for calculating the quantity of agricultural product for the area.

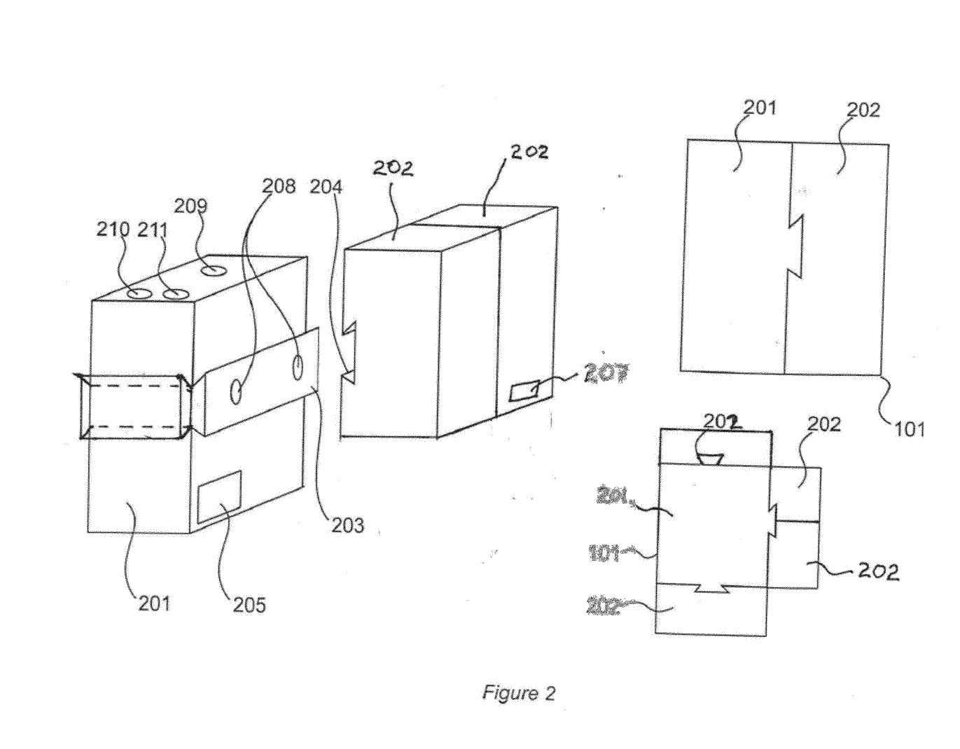

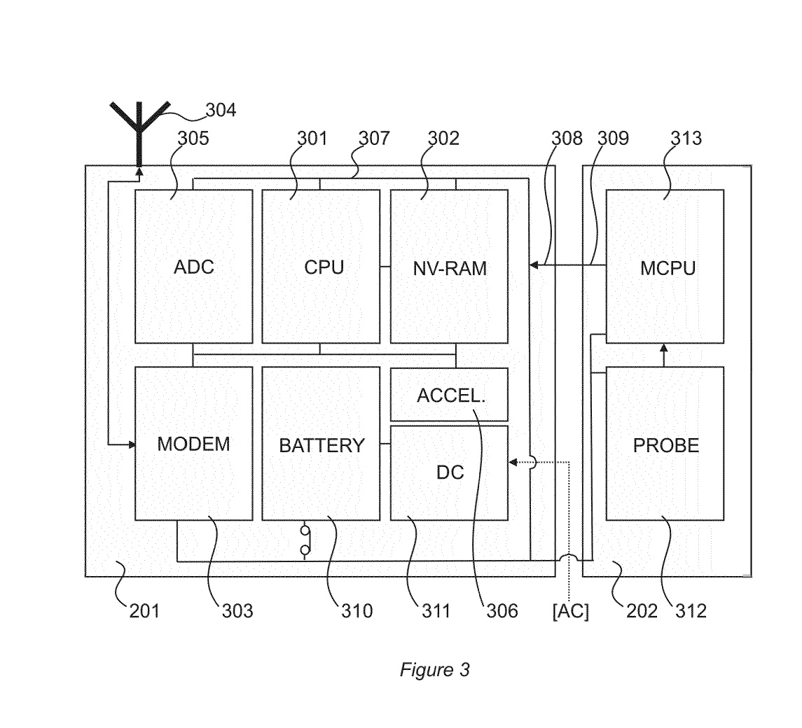

With reference to FIG. 1 and now to FIGS. 2 and 3, the accelerometer-triggered unit 101 can be manufactured as a single unit, or as a modular device comprising two or more modules 201, 202.

A first or primary module 201 is a base unit 201 which houses the accelerometer and data transmitter, whilst a secondary unit 202 houses the measuring sensor, e.g. an ultrasound range finder 207. The primary and secondary units 201, 202 are configured for releasable securing to one another and, in the example shown, are shown with corresponding tongue and groove 203, 204 respectively, wherein the tongue 203 of the primary unit 201 engages within the corresponding groove 204 of the secondary unit 202 in a sliding fit that can be then locked into place. The primary module 201 shown can be adapted to accommodate four secondary units 202 each comprising a separate sensor, as shown in a preferred embodiment, all configured to operate at the same time to provide multiple functionality in the system. It will be appreciated that the primary module 201 can be configured to receive more modules 202 depending on the number of measuring applications required.

The top side of the primary unit 201 facing the secondary unit 202 and oriented generally towards the outside of the user's boot 102 includes an optional antenna aperture or window 205, for facilitating passage of the signals transmitted by the primary unit 201 in use.

Cable or contact pads 208 on the tongue 203 of the primary module 201 and corresponding contact pads (not shown) in the groove 204 of the secondary module 202 provide a power and data bus or interface between components respectively housed in the two modules, whereby the secondary module is powered by the primary module in use.

The primary module 201 is further configured with a user interface, in the form of an on/off switch 209, a power-status led light 210 indicating the operating status of the unit 101 and a data transmission led light 211 indicating the communicating activity of the unit 101.

Respective hardware architectures of the primary and secondary modules 201, 202 suitable for measuring and communicating measurement data according to the invention are shown in FIG. 3 in further detail, by way of non-limitative example. The primary unit 201 firstly includes a data processing unit 301, for instance a general-purpose microprocessor (‘CPU’), acting as the main controller of the unit 201 and which is coupled with memory means 302, comprising non-volatile random-access memory (‘NVRAM’).

The primary unit 201 further includes a modem 303 to implement the wireless communication functionality, as the modem provides the hardware interface to external communication systems, such as the Bluetooth local wireless network 106 shown in FIG. 1. An aerial 304 coupled with the modem 303 facilitates the transmission of wireless signals to the nearby mobile data processing device 105. The modem 303 is interfaced with or includes an analogue-to-digital converter 305 (‘ADC’) for demodulating wavelength wireless signals received via the antenna 304 into digital data, and reciprocally for outgoing data. The primary unit 201 further includes an accelerometer 306.

The CPU 301, NVRAM 302, modem 303, ADC 305 and accelerometer 306 are connected by a data input/output bus 307, over which they communicate and to which further components of the handset 105 are similarly connected, in order to provide wireless communication functionality and receive measurement data. In particular, measurement data output by the secondary module 202 is received into NVRAM 302 via a connection or interface 308 between a respective data bus 309 of the secondary module 202 and the bus 306. Power is provided to the primary unit 201 by an internal battery 310, which an electrical converter 311 charges from a power supply as and when required. In one embodiment the module 201 comprises a solar cell that is configured to provide enough electrical energy to power the internal battery 310.

The secondary module 202 has a simpler architecture, since it does not carry any data transmission over a network, nor does it include a power source. Accordingly, the architecture is simplified to a maximum extent, and is limited to the sensor or probe 312 required to perform the measurement, interfaced with a microcontroller 313 which controls its operation according to triggering by the accelerometer 306 and which returns the measurement data to the NVRAM 302 of the primary unit 201.

The modular architecture of the embodiment shown in FIGS. 2 and 3 advantageously allows a multitude of secondary sensors or probes 202 to be paired with the primary module 202, thus confers a multi-purpose character to the system of the invention. For instance, such sensors 202 may include measurement sensors remote from the ground surface and adapted to determine plant height via ultrasonic emissions, to determine plant type via luminosity analysis, photosynthetically active radiation, or to determine insect infestation via spectrometric analysis. It will be appreciated that the sensors 202 can be mounted on the side of an agricultural vehicle for example an all terrain vehicle, a quad bike, a tractor or any other suitable vehicle. The sensors 202 may also include measurement sensors intended to contact the ground surface and adapted to determine soil moisture levels, silage moisture levels, soil temperature at a shallow depth, soil pH, soil/feces nitrogen content, soil phosphate content, soil lime content, methane content or soil sulphur content. For example, these sensors 202 further comprise a probe (not shown) adapted to cooperate with the soil to make measurements that are then sent to the primary module 201 for processing and/or storage.

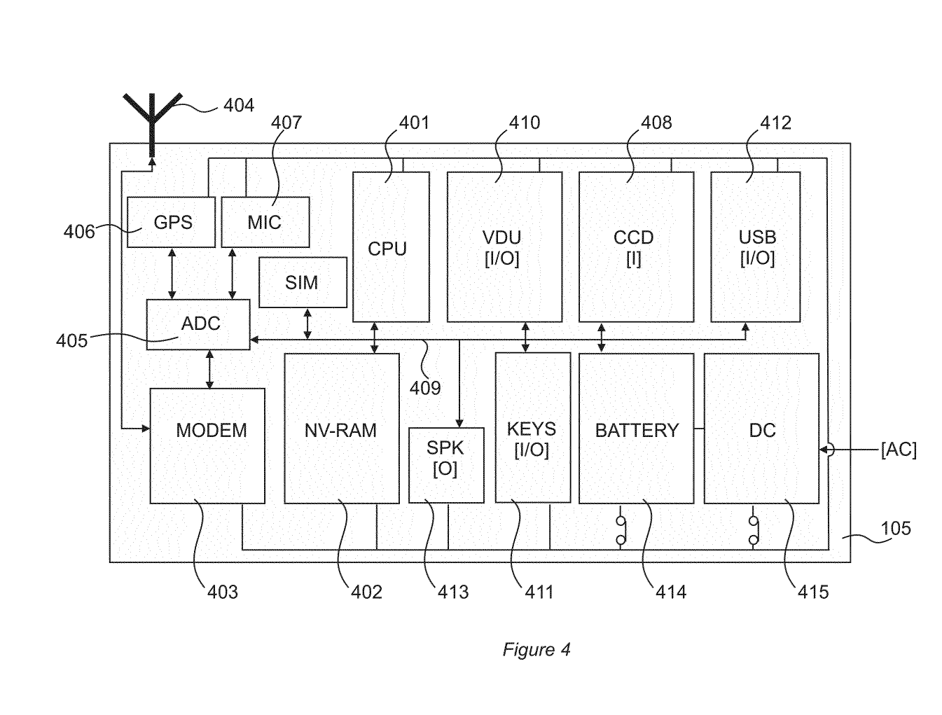

A typical hardware architecture of the mobile telephone handset 105 suitable for receiving and processing measurement data from the unit 101 according to the invention is shown in FIG. 4 in further detail, by way of non-limitative example. The handset 105 firstly includes a data processing unit 401, for instance a general-purpose microprocessor (‘CPU’), acting as the main controller of the handset 105 and which is coupled with memory means 402, comprising non-volatile random-access memory (‘NVRAM’).

The mobile telephone handset 105 further includes a modem 403 to implement the wireless communication functionality, as the modem provides the hardware interface to external communication systems, such as the GSM or GPRS cellular telephone network 107, 108, 109 shown in FIG. 1. An aerial 404 coupled with the modem 403 facilitates the reception of wireless signals from nearby communication link relays 106. The modem 403 is interfaced with or includes an analogue-to-digital converter 405 (‘ADC’) for demodulating wavelength wireless signals received via the antenna 404 into digital data, and reciprocally for outgoing data.

The handset 105 further includes self-locating means in the form of a GPS receiver 406, wherein the ADC 405 receives analogue positional and time data from orbiting satellites (not shown), which the data processing unit 401 or a dedicated data processing unit processes into digital positional and time data. The handset 105 further includes a sound transducer 407, for converting ambient sound waves, such as the user's voice, into an analogue signal, which the ADC 405 receives for the data processing unit 401 or a dedicated data processing unit to process into digital first audio data. The handset 105 may optionally further include imaging means 408 in the form of an electronic image sensor, for capturing image data which the data processing unit 401 or a dedicated data processing unit processes into digital image data.

The CPU 401, NVRAM 402, modem 403, GPS receiver 406, microphone 407 and optional digital camera 408 are connected by a data input/output bus 409, over which they communicate and to which further components of the handset 105 are similarly connected, in order to provide wireless communication functionality and receive user interrupts, inputs and configuration data.

Alphanumerical and/or image data processed by CPU 401 is output to a video display unit 410 (‘VDU’), from which user interrupts may also be received if it is a touch screen display. Further user interrupts may also be received from a keypad 411 of the handset, or from an external human interface device (‘HiD’) connected to the handset via a Universal Serial Bus (‘USB’) interface 412. The USB interface advantageously also allows the CPU 401 to read data from and/or write data to an external or removable storage device. Audio data processed by CPU 401 is output to a speaker unit 413.

Power is provided to the handset 105 by an internal module battery 414, which an electrical converter 415 charges from a suitable power supply as and when required.

It will be appreciated that the GPS functionality 406 of the mobile device 105 can be exploited to indicate where the farmer is on their land at any point in time. With the GPS 406, an application can track the farmer's movements and associate the measurement data sent back from the ultrasound device 101 with the appropriate field. With GPS integration, software and hardware aspects of the solution can work together without any need for the user's intervention.

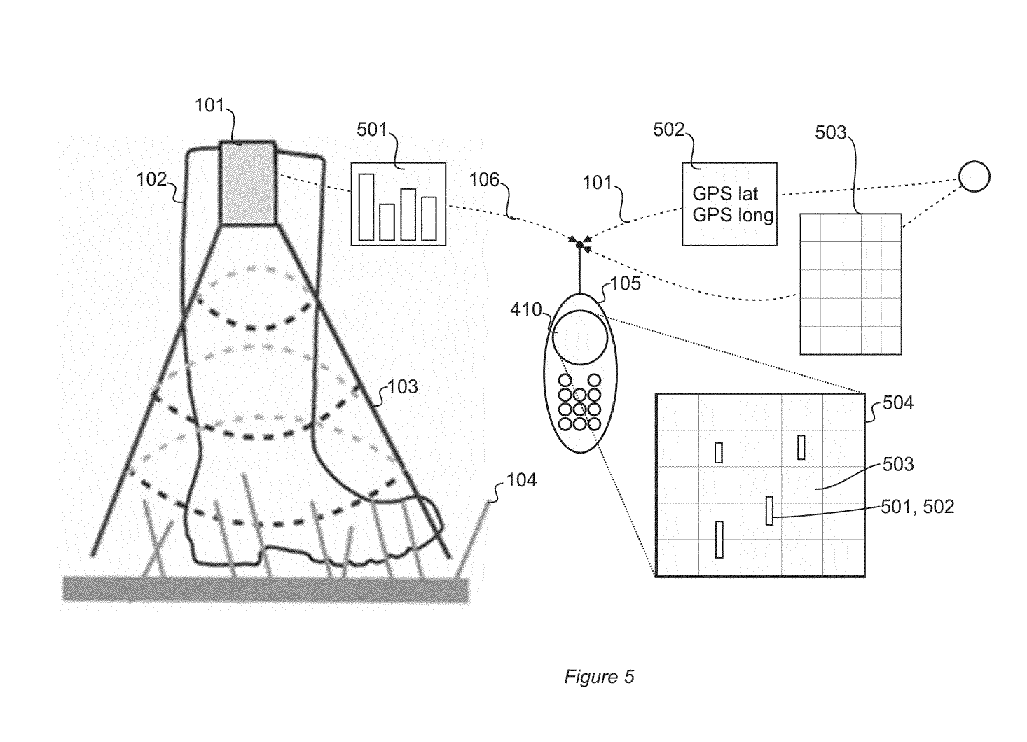

With reference to FIG. 5 now, a set of instructions stored and processed by the smart phone 105 is therefore adapted to represent the farmer's land or area as an overlay 504 on Google Maps 503 for example or other Map programs available in the market, by correlating measurement data coordinates 502 obtained with the GPS 406 therewith, and to include the measurement data 501 obtained from the accelerometer-triggered measuring unit 101 thereon.

Each field or area can be clearly defined with a translucent shape/overlay representing each field. The software application provides a means for mapping the measurement data 501 and coordinate data 502 onto a map 503. The processing part of the application calculates the quantity of measured agricultural product 501. The software application can visually represent the quantity of measured agricultural product on a colour coded map 504. The color of this overlay map indicates the status of the field. As the farmer walks his fields and paddocks The agricultural product 501 can then be calculated in real time to show the current kg DM/ha (or acre) yield per field for all of the farmer's fields.

For example, in a specific embodiment of the invention data gathered pertaining to the parameters of agriculture can be displayed in various graphical and other formats—for example grass growth could be displayed as a grass wedge, or as a bar, or pie chart, or overlaid on a map. Weather information and time and distance data can also be added. Data can also be synchronized with herd numbers and milk output. In another embodiment the results can be viewed in tabulated form and compared with previous statistical measurement over time.

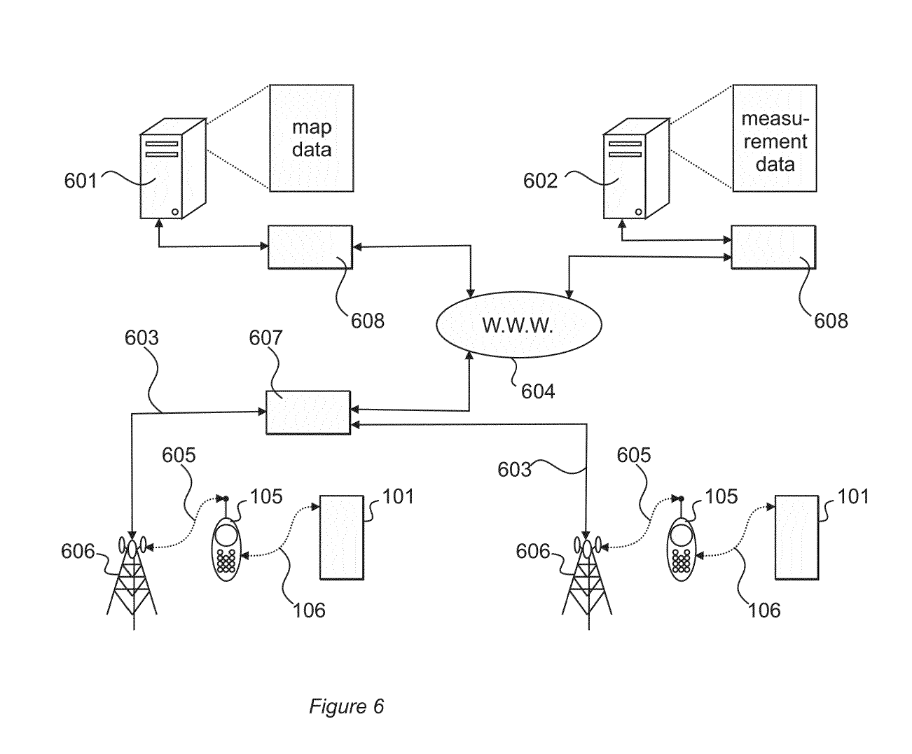

Accordingly, with reference to FIG. 6, a network environment 600 in which the system 100 may be used substantially with the features described with reference to FIG. 5, comprises a plurality of data processing terminals connected to a plurality of networks, including the mobile data processing terminal 105 and remote data processing terminal 601, 602, all interconnected via a plurality of networks 603, 604.

In the example, the mobile data processing device 105 is a mobile telephone handset 105 having wireless telecommunication emitting and receiving functionality over a cellular telephone network 603 configured according to the Global System for Mobile Communication (‘GSM’), General Packet Radio Service (‘GPRS’), International Mobile Telecommunications-2000 (IMT-2000, ‘W-CDMA’ or ‘3G’) network industry standards, and wherein telecommunication is performed as voice, alphanumeric or audio-video data using the Short Message Service (‘SMS’) protocol, the Wireless Application protocol (‘WAP’) the Hypertext Transfer Protocol (‘HTTP’) or the Secure Hypertext Transfer Protocol (‘HTTPS’).

The mobile telephone handset 105 receives or emits voice, text, audio and/or image data encoded as a digital signal over a wireless data transmission 605, wherein the signal is relayed respectively to or from the handset by the geographically-closest communication link relay 606 of a plurality thereof. The plurality of communication link relays 606 allows digital signals to be routed between the handset 105 and their destination by means of a remote gateway 607 via a MSC or base station. The gateway 607 is for instance a communication network switch, which couples digital signal traffic between wireless telecommunication networks 603, such as the cellular network within which wireless data transmissions 605 take place, and a Wide Area Network (WAN) 604. The gateway 607 further provides protocol conversion if required, for instance whether a handset 105 uses the WAP or HTTPS protocol to communicate data.

Remote data processing terminals 601, 602 are conventional desktop computers or servers 601, 602, each of which emits and receives data encoded as a digital signal over a wired data transmission conforming to the IEEE 802.3 (‘Gigabit Ethernet’) standard, wherein the signal is relayed respectively to or from the computer by a wired router 608 interfacing the computer 601, 602 to the WAN communication network 604. In the example, computer 601 is a server storing and distributing pre-configured map data 503 to requesting nodes across the network 604, for instance a server operated by the Google Corporation and configured to distribute the GoogleMaps data described with reference to FIG. 5.

It will be appreciated that the application of the invention has the capability to export the collated data to a third party and allows the user to share the data with other farmers. Accordingly, in the example still, computer 602 is a desktop computer operated by a user tasked with collating respective agricultural products measurement data from users of systems 100, for instance a dairy plant requiring such data for milk output planning, or a government agency requiring such data for statistical purposes.

It will be appreciated that the measured data can be linked to GPS, date and weather, as well as stocking rates, herd details, input, fertilizer and feed details to provide an accurate picture of the farm at the touch of a button.

It will be further appreciated that while the present invention describes an application to grass the invention can also be applied to calculating the yields of other agricultural crops for calculation of their respective yields using the same system and method as hereinbefore described. In another embodiment the invention can be used for calculating quantities of uncut grass on a golf course or other public park area.

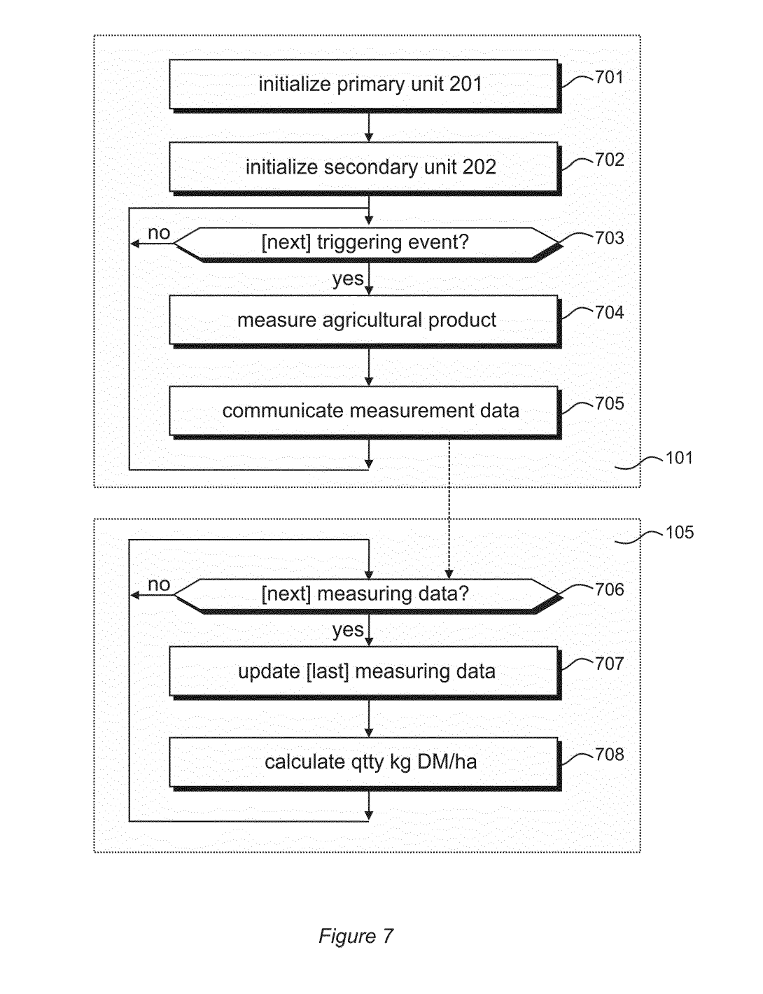

With reference to FIG. 7 now, the core methodology implemented by the set of instructions in the memory 302 of the primary module 201 and the memory 402 of the mobile phone handset 105 comprises an initialization of the primary module 201 at step 701 and an initialization of the secondary module 201 at step 702. At step 703, a question is asked as to whether a triggering input or pulse has been received from the accelerometer 306. If the question is answered negatively, the logic loops until an accelerometer input or impulse is received. As and when the input or pulse is received, then at step 704 the primary module triggers the sensor 312 of the secondary module 202 via its microcontroller 313 and receives a corresponding measurement data in return, which it communicates to the mobile phone handset 105 at step 705. The logic returns to the question of step 703, whereby a next input or pulse may trigger a next measurement, and so on and so forth.

In parallel, the set of instructions stored and processed by the mobile handset 105 implements a logic, pursuant to which the phone polls the local network for signals from the primary module 201 containing measurement data, shown as a question step 706. Similarly to the question of step 703, the logic loops until such time as a measurement data signal is received. As and when such a signal is received, then the application updates the measurement data 501 already stored therein therewith at step 707, and re-calculates the cumulative quantity of dry matter for the expanding area of measurement at step 708. The logic returns to the question of step 706, whereby a next measurement data signal may trigger a next update and calculation, and so on and so forth.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

It will be appreciated that the invention can be used to provide data for pesticide applications, fertiliser applications, water management, pruning decision making, re-seeding and all other crop management decisions.

It will be appreciated that in the context of the present invention the quality of a crop encompasses sensory attributes, nutritive values, chemical constituents, mechanical properties, functional properties and defects. Instrumental measurements are often preferred to sensory evaluations in research and commercial situations because they reduce variations in judgment among individuals and can provide a common language among researchers, industry and consumers. Essentially, electromagnetic (often optical) properties relate to appearance, mechanical properties to texture and growth rate, and chemical properties to flavour (taste and aroma).

The invention is not limited to the embodiments hereinbefore described but may be varied in both construction and detail.

1. A system for measuring a quantity of an agricultural product, the system comprising:
means for measuring agricultural product data;
an accelerometer for triggering the measuring means and repeating the measurement at a plurality of locations over an area;
means for communicating the measurement data to a remote processing means; and
means for calculating the quantity of agricultural product for the area using the processing means.

2. The system as claimed in claim 1, wherein the accelerometer and the communicating means are contained in a primary module having a rechargeable power source.

3. The system as claimed in claim 1, wherein the means for measuring data are contained in a secondary module drawing power from a primary module.

4. The system as claimed in claim 1, wherein a primary module is configured to releasably retain the secondary module in use.

5. The system as claimed in claim 1, wherein a primary module is configured to releasably retain a plurality of secondary modules in use.

6. The system as claimed in claim 1, wherein the means for measuring data is selected from the group comprising an ultrasonic ranging device, a luminosity measuring device, a photosynthetically active radiation meter and a spectrometer and
wherein the at least one ultrasonic ranging device is adapted to measure a plant height in the range 0 to 1.5 cm, or in the range 1.5 to 30 cm, or in the range 30 to 150 cm.

7. (canceled)

8. The system as claimed in claim 1, wherein the means for communicating the measurement data is selected from the group comprising a near field communication device according to the ISO 13157 networking standard, a wireless data transmitter conforming to the IEEE 802.15.1 Bluetooth networking standard, a wireless data transmitter conforming to the IEEE 802.11 WiFi networking standard.

9. The system as claimed in claim 1, wherein the remote processing means further comprises means for associating measurement data with coordinate data and
wherein the remote processing means further comprises means for mapping coordinate data and measurement data to define an measured area.

10. (canceled)

11. The system as claimed in claim 1, wherein the remote processing means further comprises means for associating measurement data with coordinate data and wherein the remote processing means further comprises means for mapping associated measurement data and coordinate data on a map.

12. The system as claimed in claim 1, further comprising an interface for visually representing the quantity of measured agricultural product on a colour-coded map.

13. (canceled)

14. The system as claimed in claim 1, wherein the remote processing means is selected from the group comprising computers, portable computers, tablet computers, mobile telephone handsets.

15. The system as claimed in claim 1, further comprising a mounting bracket for mounting the system to a support, at a height substantially above the agricultural product to be measured and wherein the mounting bracket is one suitable for a support selected from the group comprising user footwear, a walking aid, a post, a gate, a fence, a user vehicle, a mobile robot, a drone aerial vehicle, an animal.

16-17. (canceled)

18. A method for measuring a quantity of an agricultural product, for example grass, the method comprising the steps of:
measuring the agricultural product upon receipt of a measurement trigger provided by an accelerometer;
repeating the measurement trigger at a plurality of locations over an area;
communicating the measurement data to a remote processing means; and
calculating the quantity of agricultural product for the area at the remote processing means.

19. The method as claimed in claim 18, wherein the step of measuring is performed by at least one selected from the group comprising an ultrasonic ranging device, a luminosity measuring device, a photosynthetically active radiation meter and a spectrometer.

20. The method as claimed in claim 19, wherein the step of measuring performed by the at least one ultrasonic ranging device further comprises measuring a plant height in the range 0 to 1.5 cm, or in the range 1.5 to 30 cm, or in the range 30 to 150 cm.

21. The method as claimed in claim 18, wherein the step of communicating the measurement data is performed by one selected from the group comprising a near field communication device according to the ISO 13157 networking standard, a wireless data transmitter conforming to the IEEE 802.15.1 Bluetooth networking standard, a wireless data transmitter conforming to the IEEE 802.11 WiFi networking standard.

22. The method as claimed in claim 18, comprising the further step of associating measurement data with coordinate data and comprising the further step of mapping coordinate data and measurement data to define a measured area.

23. (canceled)

24. The method as claimed in claim 18, wherein the remote processing means further comprises means for mapping associated measurement data and coordinate data on a map and further comprising an interface for visually representing the quantity of measured agricultural product on a colour-coded or gray-scale map.

25-26. (canceled)

27. The method as claimed in claim 18, wherein the remote processing means is selected from the group comprising computers, portable computers, tablet computers, mobile telephone handsets.

28. The method as claimed in claim 18, comprising the further step of mounting the system to a support, at a height substantially above the agricultural product to be measured.

29-33. (canceled)
